(19) United States
(12) Patent Application Publication
(10) Pub. No.: US 2026/0167971 A1
(43) Pub. Date: Jun. 18, 2026

(54) Articles and Methods for Treating Renal Fibrosis

(71) Applicant: Vanderbilt University, Nashville, TN (US)

(72) Inventors: William D Merryman, Nashville, TN (US); Craig L. Duvall, Nashville, TN (US); Tessa Huffstater, Nashville, TN (US); Ella Hoogenboezem, Nashville, TN (US)

(21) Appl. No.: 19/122,599

(22) PCT Filed: Oct. 19, 2023
(86) PCT No.: PCT/US2023/077350

§ 371 Date: Apr. 18, 2025

Related U.S. Application Data
(60) Provisional application No. 63/417,432, filed on Oct. 19, 2022.

(51) Int. Cl. C12N 15/113; A61K 31/712; A61K 31/7125; A61K 31/713; A61P 13/12

(52) U.S. Cl. CPC C12N 15/1138; A61K 31/712; A61K 31/7125; A61K 31/713; A61P 13/12; C12N 2310/14; C12N 2310/315; C12N 2310/321; C12N 2310/322

(57) ABSTRACT

A method of treating, ameliorating, or preventing a condition characterized by one or both of reduced renal function and renal fibrosis is described, which includes interfering with one or both of an expression and an activity of a cadherin. In embodiments, the kidney disease is chronic kidney disease (CKD). The interfering may include reducing or preventing the expression of the cadherin. The cadherin may be cadherin-11 (CDH-11), and the reducing or preventing the expression thereof may be achieved by a specific inhibition or knockdown of gene expression of CDH-11 via a modified silencing RNA (siRNA) having an improved nuclease stability.

### RELATED APPLICATIONS

This is a utility patent application claiming the benefit of priority in U.S. Provisional Application Ser. No. 63/417,432 filed Oct. 19, 2023, the entire disclosure of which is incorporated herein by reference.

### GOVERNMENT INTEREST

This invention was made with government support under Grant No. HL135790 awarded by the National Institutes of Health. The government has certain rights in the invention.

### TECHNICAL FIELD

The presently-disclosed subject matter relates to articles and methods for preventing renal fibrosis. In particular, certain embodiments of the presently-disclosed subject matter relate to zipper modified siRNA that knockdown cadherin-11 and methods of use thereof to treat renal fibrosis.

### BACKGROUND

Chronic kidney disease (CKD) is a condition characterized by progressive damage and loss of function in the kidneys. CKD is defined by reduced kidney function for >3 months, often leading to end-stage renal disease (ESRD), which requires dialysis or transplant. Kidney injury leads to a cascade of molecular events that recruit inflammatory cells, activate myofibroblasts, and attempt to regenerate tubular epithelial cells. Prolonged occurrence of these events leads to fibrosis and inflammation which are the hallmarks of CKD. Moreover, persistent tubular injury leads to de-differentiation of tubular epithelial cells and activation of myofibroblasts and inflammatory cell recruitment. Once tubular epithelial cells are injured or lost, CKD progression is rapid and degenerative.

Treatment of CKD is a massive economic burden on the US health care system, affecting more than 30M Americans and costing Medicare over $79B per year. While CKD and ESRD patients make up just 13% of the Medicare population, their treatment constitutes 23% of the total cost of Medicare. Today, over 661k Americans have ESRD and many more are on their way with early stage CKD. The CDC estimates that nearly half of all 30-year-olds will develop CKD in their lifetime, 80% of these will develop stage 3 to 5 CKD, and 11% of those who reach stage 3 will end up with kidney failure. These numbers will only increase as the population ages and there remain no curative therapies.

While there are multiple etiologies of CKD, the vast majority of cases are caused by HTN and/or diabetes, and in turn, patients with CKD are much more likely to develop cardiovascular disease, which is the leading cause of death worldwide. Thus, new treatment strategies for CKD are urgently needed.

### SUMMARY

In accordance with the purposes and benefits described herein, a method for treating, ameliorating, or preventing a condition characterized by one or both of reduced renal function and renal fibrosis is disclosed, comprising interfering with one or both of an expression and an activity of a cadherin. In embodiments, the kidney disease is chronic kidney disease (CKD) and the interfering comprises reducing or preventing the expression of the cadherin. In embodiments, the cadherin is cadherin-11 (CDH-11).

In one possible embodiment, the reducing or preventing the expression of CDH-11 is achieved by a specific inhibition or knockdown of gene expression of CDH-11 using a silencing RNA (siRNA). In embodiments, the siRNA is modified to improve a nuclease stability. In embodiments, the modified siRNA exhibits improvements in one or both of an endonuclease stability and an exonuclease stability. In other embodiments, in the modified siRNA phosphothorothioate bonds are substituted for phosphodiester bonds in the siRNA strand ends.

In still yet other embodiments, the modified siRNA is modified at a 2′ position of a ribose ring. The modifications may include one or both of a 2′-fluoro (2′F) modification and a 2′-O-methyl(2′Ome) modification.

In one embodiment, the modified siRNA comprises alternating 2′-fluoro (2′F) and 2′-O-methyl(2′Ome) modifications (zip-siRNA). In a particular embodiment, the zip-siRNA is a 19mer duplex termed zip-siCDH11 having a formula:

Antisense:
5′ (PHO)(MeU)*(fA)*(MeU)(fC)(MeA)(fC)(MeA)(fA)
(MeA)(fG)(MeA)(fA)(MeU)(fU)(MeG)(fG)(MeU)*(fU)
*(MeC) 3′
Sense:
5′ (fG)*(MeA)*(fA)(MeC)(fC)(MeA)(fA)(MeU)(fU)
(MeC)(fU)(MeU)(fU)(MeG)(fU)(MeG)(fA)*(MeU)*(fA) 3′
wherein f = 2′F; Me = 2′-O-Me; * = PS bond; and
PHO = phosphate.

In another aspect, a method of treating, ameliorating, or preventing a condition characterized by one or both of reduced renal function and renal fibrosis is described, comprising administering an inhibitor of cadherin expression. The kidney disease may be chronic kidney disease (CKD).

In one possible embodiment, the cadherin is cadherin-11 (CDH-11) and the inhibitor is a silencing RNA (siRNA). The siRNA may be modified to improve a nuclease stability, in particular embodiments being one or both of an endonuclease stability and an exonuclease stability.

In another possible embodiment, in the modified siRNA phosphothorothioate bonds are substituted for phosphodiester bonds in the siRNA strand ends.

In particular embodiments, the modified siRNA is modified at a 2′ position of a ribose ring. The modified siRNA may be modified to include one or both of a 2′-fluoro (2′F) modification and a 2′-O-methyl(2′Ome) modification.

In one possible embodiment, the modified siRNA comprises alternating 2′-fluoro (2′F) and 2′-O-methyl(2′Ome) modifications (zip-siRNA).

In one particular embodiment, the zip-siRNA is a 19mer duplex termed zip-siCDH11 having a formula:

Antisense:
5′ (PHO)(MeU)*(fA)*(MeU)(fC)(MeA)(fC)(MeA)(fA)
(MeA)(fG)(MeA)(fA)(MeU)(fU)(MeG)(fG)(MeU)*(fU)*
(MeC) 3′
Sense:
5′ (fG)*(MeA)*(fA)(MeC)(fC)(MeA)(fA)(MeU)(fU)(MeC)
(fU)(MeU)(fU)(MeG)(fU)(MeG)(fA)*(MeU)*(fA) 3′
wherein f = 2′F; Me = 2′-O-Me; * = PS bond; and
PHO = phosphate.

In yet another aspect of the disclosure, a method of treating, ameliorating, or preventing a condition characterized by one or both of reduced renal function and renal fibrosis is described, comprising administering zip-siCDH11 to a patient in need thereof in an amount sufficient to reduce or prevent cadherin-mediated suppression of alpha-1-antitrypsin (AAT) synthesis;
wherein zip-siCDH11 is a 19mer duplex termed zip-siCDH11 having a formula:

CDH11Antisense:
5′ (PHO)(MeU)*(fA)*(MeU)(fC)(MeA)(fC)(MeA)(fA)
(MeA)(fG)(MeA)(fA)(MeU)(fU)(MeG)(fG)(MeU)*(fU)*
(MeC) 3′
CDH11Sense:
5′ (fG)*(MeA)*(fA)(MeC)(fC)(MeA)(fA)(MeU)(fU)(MeC)
(fU)(MeU)(fU)(MeG)(fU)(MeG)(fA)*(MeU)*(fA) 3′
wherein f = 2′F; Me = 2′-O-Me; * = PS bond; and
PHO = phosphate.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description refers to the following drawings, wherein:

FIGS. 1A-B show Gene expression at 7 days following UUO in Cdh11 mutant mice (A), C57BL/6 mice pretreated with CDH11 mAb, SYN0012 (B). N shown inside bars; *p<0.05, ***p<0.001, *****p<0.00005 vs Control or Cdh11^{+/+}.

FIGS. 2A-G show Survival (A), BUN (B), plasma IL-6 (C), histology (D-F), and stiffness (G) in Cdh11 mutant animals injured with AA. *p<0.05 and **p<0.005 vs Cdh11^{+/+}.

FIGS. 3A-D show BUN at 7 days (A) and histology at 28 days (B-D) in C57BL/6 mice injured with AA and treated with IgG2a vs SYN0012, a mAb against CDH11. *p<0.05 vs IgG.

FIGS. 4A-D show BP (A) and BUN (B) of HTN model (uninephrectomy and AngII) of kidney injury in CDH11 mutant mice. CDH11 is in proximal tubules of WT mice at day 28 of the HTN model (C and D), while none detected in CDH11^{-/-} mice. *p<0.05 vs Cdh11^{+/+} Unix&AngII.

FIGS. 5A-B show BP (A) and ACR (B) in mice exposed to the HTN model for 28 days while treated with a mAb targeting of CDH11 (SYN0012). *<0.05 vs IgG.

FIGS. 6A-B show Human kidney tissue specimens classified as “severe fibrosis” (A) and healthy control (B).

FIG. 7 shows RNAseq analysis reveals that Serpina1d, the gene for AAT, is the most significantly increased gene in both AA and HTN injury models in Cdh11 null mice. Red=increased in Cdh11 null mice; blue-increased in WT mice. N=4 for every treatment/genotype, except WT HTN (N=3).

FIG. 8 shows 72 h of siCDH11 in uninjured, immortalized PTECs increases AAT 2.2-fold.

FIG. 9 shows cumulative incidence of CKD increases with Z alleles of SERPINA1, the gene for AAT. Adjusted p value for one Z allele is 1.77×10⁻⁶ and for two Z alleles is 2.97×10⁻⁹.

FIG. 10 shows protein-protein interactions revealed from HitPredict linking CDH11 and AAT (left). 10 of the 51 genes that were significantly changed in both AA and HTN RNAseq dataset interact directly with CDH11, β-catenin, ESR2, AAT pathway (right).

FIG. 11 shows alternative targets, C3 and Fgl1.

FIGS. 12A-B show mAb targeting of CDH11 beginning after 28 days of HTN model—with treatment continuing for 8 weeks—shows promising trends with reduced ACR (A) and inflammatory/fibrosis markers (B). *p<0.05.

FIGS. 13A-E show zip-siRNA design strategy (A). These zip-siRNAs are not digested in serum like other siRNAs (B) and preferentially home to kidneys (C, D) and PTECs (E) 1 h after injection.

FIGS. 14A-C show siRNA in vitro optimization in cardiac fibroblasts, which express high levels of CDH11, to identify siCDH11-3 (A) to construct zip-siCDH11, which maintained knockdown in the same fibroblasts (B). Zip-siCDH11 reduces kidney CDH11 expression in vivo by 40% 48 h after UUO and increases AAT by 80% (C).

FIGS. 15A-B show treating CKD mice (HTN model for 4 weeks) with CDH11 mAb (SYN0012) prevents LV mass increase (A) and heart failure marker BNP (B). n≥5 for all data points. **p<0.001. *p<0.05.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter relates to articles and methods for treating kidney disease. The present inventors have identified cadherin-11 (CDH11) as a mediator of kidney disease that can be targeted for therapy. For example, the present inventors have found that CDH11 is expressed on the proximal tubule epithelial cells (PTECs) when these cells become injured in both human cases and mouse models of kidney fibrosis. Additionally, the present inventors have found that CDH11 expression inversely correlates with expression of alpha-1-antitrypsin (AAT), which as been shown to be protective in models of kidney fibrosis. Accoringly, in some embodiments, the method includes targeting cadherin-11 (CDH11) as a mediator of kidney disease. For example, in one embodiment, the method includes reducing or preventing expression of CDH11. In another embodiment, the method includes administering siRNA to reduce or prevent expression of CDH11. In some embodiments, the method may be used to treat any suitable kidney disease, such as, but not limited to, kidney fibrosis, chronic kidney disease, or any other kidney disease exhibiting increased expression of CDH11.

Also provided herein is a modified RNA with improved nuclease stability. In some embodiments, the modified RNA includes a zipper modified siRNA (zip-siRNA). In some embodiments, the zip-siRNA includes siRNA with a “zipper” pattern that uses alternating 2′-fluoro (2′F) and 2′-O-methyl(2′Ome) modifications. In some embodiments, the modifications to form the zip-siRNA are on blunt-ended 19-base-pair duplex siRNA sequences. The zip-siRNA according to one or more embodiments disclosed herein provides increased endonuclease stability while maintaining target gene silencing activity relative to a parent unmodified RNA version of the sequence. Additionally or alternatively, in some embodiments, the siRNA strand ends are modified to contain phosphorothioate bonds rather than phosphodiester. In some embodiments, the end modifications protect against endonucleases. Although described herein primarily with respect to a zipper pattern with 2′F and 2′Ome modifications, as will be appreciated by those skilled in the art, the disclosure is not so limited and any other suitable modification or pattern to increase nuclease stability is contemplated. In some embodiments, the zip-siRNA homes to the kidneys. Additionally or alternatively, in some embodiments, the zip-siRNA can be used to knockdown CDH11 and enhance AAT expression in kidney fibrosis. Accordingly, in some embodiments, the method of treating kidney disease includes administering the zip-siRNA to a subject in need thereof.

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below. While the terms used herein are believed to be well understood by those of ordinary skill in the art, certain definitions are set forth to facilitate explanation of the presently-disclosed subject matter.

The terms “polynucleotide”, “nucleic acid”, or “oligonucleotide” as used herein, refer to a polymer of nucleotides. The terms “polynucleotide”, “nucleic acid”, and “oligonucleotide”, may be used interchangeably. Typically, a polynucleotide comprises at least three nucleotides. DNAs and RNAs are exemplary polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thithymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The polymer may also be a short strand of nucleic acids such as RNAi, siRNA, or shRNA.

The terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The terms “treatment” or “treating” refer to the medical management of a patient with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. As will be recognized by one of ordinary skill in the art, the term “cure” does not refer to the ability to completely eliminate a disease or condition. For example, in some embodiments, a cure can refer to a decrease at a level of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. Similarly, as will be recognized by one of ordinary skill in the art, the term “prevent” does not refer to an ability to completely remove any and all symptoms or evidence of a disease or condition.

Likewise, as will be recognized by one of ordinary skill in the art, the term “inhibiting” or “inhibition” does not refer to the ability to completely inactivate all target biological activity in all cases. Rather, the skilled artisan will understand that the term “inhibiting” refers to decreasing biological activity of a target, such as METAP, such as can occur, for example, when a nucleotide limits the expression of the target gene, when a ligand binding site of the target protein is blocked, or when a non-native complex with the target is formed. Such decrease in biological activity can be determined relative to a control, wherein an inhibitor is not administered and/or placed in contact with the target. For example, in some embodiments, a decrease in activity relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. The term “inhibitor” refers to a compound of composition that reduces the expression of and/or decreases the biological activity of a target.

The term “administering” refers to any method of providing a therapeutic composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable means such as intravenous administration, intra-arterial administration, intramuscular administration, peritoneal administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention or amelioration of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9): 1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### Examples

Cadherin-11 (CDH11) was recently identified as a potential biomarker of renal fibrosis. CDH11 is expressed by injured epithelial, mesenchymal, and activated hematopoietic cells, including neutrophils, macrophages, and monocytes. It has been found to be a driver of rheumatoid arthritis and aortic valve disease through regulation of fibroblast cell-cell adhesion⁸ and pro-inflammatory signaling in these tissues, believed to arise due to ‘cadherin-switching’ (i.e. N-cadherin is lost as CDH11 is expressed). The present inventors have recently shown that targeting CDH11—genetically or pharmacologically—improves heart function after a myocardial infarction and reduces fibrotic signaling, allowing for adequate scar healing. Additionally, data from multiple groups have revealed that targeting CDH11 results in reduced fibrotic signaling; however, the mechanism by which CDH11 engagement between cells leads to inflammation/fibrosis is still unknown.

In this Example, the present inventors demonstrate that CDH11 expression in injured proximal tubule epithelial cells (PTECs) is a direct mediator of CKD progression and propose that it can be targeted as a therapy for renal fibrosis. CDH11 is a homotypic cell-cell adhesion protein whose bonds are twice as strong as all other cadherins. To assess the potential of targeting CDH11 to prevent/treat CKD, we have examined three kidney injury models-unilateral ureter obstruction (UUO), aristolochic acid (AA), and a hypertension (HTN) model of uninephrectomy and angiotensin II pump implantation. These models reveal that CDH11 is exclusively expressed in the PTECs only after injury, but not elsewhere in the kidneys. Moreover, in all three injury models, genetic loss of Cdh11 or mAb targeting of CDH11 leads to improved renal function. To determine the mechanism by which targeting CDH11 improves CKD, we performed RNAseq on kidneys from AA and HTN injury models and found that loss of Cdh11 leads to high expression of Serpina1d, which encodes for alpha-1-antitrypsin (AAT), a protease inhibitor with a range of activity including inhibition of inflammatory and apoptotic enzymes that has been shown to be beneficial in preventing kidney injury. Without wishing to be bound by theory, it is believed that targeting CDH11 prevents de-differentiation and promotes survival of PTECs, partially through increased AAT expression, and is a novel therapeutic strategy for treating CKD. The information discussed herein addresses the dearth of therapeutics that alter the pathophysiology of tubular epithelial cell de-differentiation and progression of CKD.

Determining the Mechanism by which CDH11 Engagement Between PTECs Leads to Tubulo-Interstitial Fibrosis and CKD.

Without wishing to be bound by theory, it is believed that PTECs mechanically engage via homotypic CDH11 bonds early in kidney injury, leading to de-differentiation of PTECs and fibrosis over time through loss of AAT expression.

Importance: CKD is defined by reduced kidney function for >3 months, often leading to end-stage renal disease (ESRD), which requires dialysis or transplant. Today, over 661k Americans have ESRD and many more are on their way with early stage CKD. The CDC estimates that nearly half of all 30-year-olds will develop CKD in their lifetime, 80% of these will develop stage 3 to 5 CKD, and 11% of those who reach stage 3 will end up with kidney failure. These numbers will only increase as the population ages and there remain no curative therapies. Treatment of CKD is a massive economic burden—while CKD and ESRD patients make up just 13% of the Medicare population, their treatment constitutes 23% of the total cost of Medicare. While there are multiple etiologies of CKD, the vast majority of cases are caused by HTN and/or diabetes, and in turn, patients with CKD are much more likely to develop cardiovascular disease, which is the leading cause of death worldwide. Thus, new treatment strategies for CKD are urgently needed.

Critical barriers: There are no pharmacological therapies that halt progression of CKD to ESRD, though some do slow the process. Regardless of the initial cause of CKD, renal fibrosis and excessive inflammatory cytokine activity are common pathological features of disease progression. Recent data show that reducing macrophage infiltration and lymphocyte depletion prevents fibrosis in acute to chronic kidney injury models. Therefore, the inflammatory signals that emerge with kidney injury are thought to be a key to developing new renal-specific therapies for CKD. Two of the primary targets for many groups working towards therapy for CKD are TGF-β1 and IL-6, both of which play a significant role in fibrotic disease progression in the kidney. However, targeting either of these pathways specifically is complicated by their homeostatic function in normal tissue repair and the inability to localize therapies to the kidney specifically. Without wishing to be bound by theory, it is believed that there are upstream proteins in the PTECs that may be targeted to both prevent the synthesis of these inflammatory molecules and de-differentiation of the PTECs as CKD progresses. Though CDH11 has been identified as a biomarker of CKD and exogenous delivery of AAT is beneficial in models of kidney injury, little is known about these proteins, which are the foci of this Example.

Impact of project on CKD: With respect to the mechanism of CDH11 in the context of CKD, and with the proximal tubule having recently been recognized as a key player in renal fibrosis, it is believed that CDH11 engagement between PTECs leads to loss of AAT expression and other epithelial hallmarks, such as N-cadherin, which results in PTECs undergoing de-differentiation. Additionally, besides the role of CDH11 in progression of CKD, examination of intercellular cadherin biology in the kidney in general is novel and may indicate a new mechanism by which proximal tubule damage is governed by cell-cell communication, possibly through cadherin switching which may control the expression of AAT. Furthermore, if the effect of targeting CDH11 in the PTECs is not the mechanism of action, an alternative mechanism may be where targeting CDH11 prevents inflammatory cell recruitment to the kidney and interaction with PTECs via CDH11, which reduces fibrosis and preserves kidney function. Finally, a new CKD therapeutic strategy (i.e. zip-siCDH11) is being compared to a mAb that the present inventors have used extensively in their preliminary data and that has undergo Phase IIb clinical trials for rheumatoid arthritis. It is believed that this comparison is very important as IgG mAbs are extremely long circulating due to the interaction of the Fc region with the recycling receptor that leads to re-adsorption in the blood stream rather than deposition in the kidney. Since zip-siRNAs do not have a re-adsorption mechanism, they accumulate heavily in the kidneys, providing for a natural drug delivery strategy to treat renal fibrosis by targeting CDH11 in PTECs.

Again, without wishing to be bound by theory, it is believed that targeting CDH11 to prevent PTEC de-differentiation and preserve AAT expression is a novel mechanistic therapeutic strategy for CKD. Moreover, the need for new strategies to treat CKD, combined with a mAb for CDH11 that has been safely tested in humans previously, provides us with an accelerated potential pathway to the clinic for this target. Finally, it is believed that zip-siRNA targeting of CDH11 may be superior to mAb treatment and this could result in a ‘first in class’ renal-specific drug delivery strategy for CKD.

### Targeting CDH11 for CKD

This section presents the concept of targeting CDH11 for CKD, which represents a novel molecular approach and possibly a clinical innovation.

CDH11: The present inventors previously discovered that CDH11 was required to develop calcific nodules in vitro with heart valve fibroblasts; moreover, they found that it is enriched in calcified human valves. They have also shown that CDH11 is inversely expressed with N-cadherin and targeting CDH11 in a genetic model of calcific aortic valve disease prevents every hallmark of the disease. The present inventors further showed the beneficial effects of CDH11 targeting after myocardial infarction (MI). As with aortic valve disease, targeting CDH11 in a mouse model of MI reduced inflammation in the infarcted region at three days post-MI and ultimately led to better outcomes eight weeks after MI, such as reduced left ventricle volume, improved ejection fraction, and less scar expansion into the healthy myocardium.

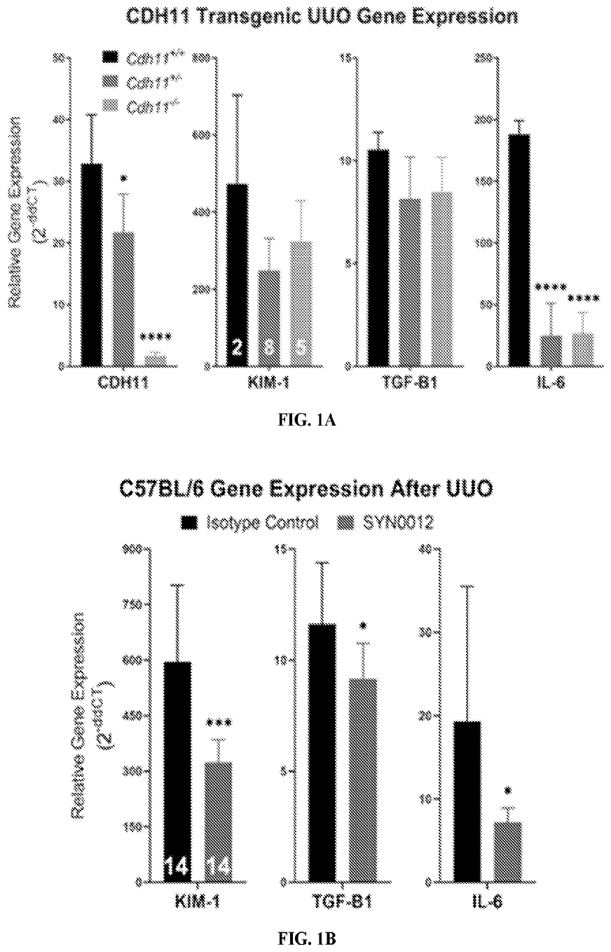

Targeting CDH11 in the UUO kidney injury model: With respect to CKD, the present inventors initially examined the potential role of CDH11 in CKD with the UUO model, wherein one kidney is obstructed with a ureteral ligation, leading to rapid progression of CKD. Seven days after the UUO procedure, the animals were sacrificed and we examined common inflammatory markers. Here, we found that IL-6 transcription was significantly reduced in the kidneys of the Cdh11 mutant animals, with there being no difference between the null and het groups (FIG. 1A), indicating that total loss of Cdh11 is not required to see a marked decrease in IL-6. Having found that genetic deletion of Cdh11 decreases inflammatory markers, we next tested a functional blocking mAb against CDH11 (i.e. SYN0012, provided by Roche) that we have used previously in cardiovascular models of disease. Because this model is so rapid and destructive, SYN0012 injection was given one week before the UUO procedure, and in this case we saw significant decreases in kidney injury marker-1 (KIM-1), TGF-β1, and IL-6 expression versus isotype control (FIG. 1B). These data indicated to us that targeting CDH11—genetically or pharmacologically—reduces inflammation and the severity of injury in the UUO mouse model.

Targeting CDH11 in the AA kidney injury model: To determine if global mutation of Cdh11 had any impact on survival in a mouse model that is considered more like an acute kidney injury (AKI) that leads to CKD, we used the AA injury model (i.e. 2-3 mg/kg AA injections daily for five days). We found that all Cdh11^{-/-} mice survived the injury while 25% of Cdh11^{+/+} died before the 28 day endpoint (FIG. 2A); moreover, at seven days after their final injection of AA, Cdh11 null animals had significantly lower blood urea nitrogen (BUN) compared to WT mice (FIG. 2B) and circulating plasma levels of IL-6 were reduced, though not significantly in this preliminary analysis (FIG. 2C). Note that these differences are likely underestimates since 15% of Cdh11^{+/+} died before the seven day time point when BUN was measured (FIG. 2A). Tissue morphology was preserved in the mutant kidneys with less fibrosis and tubular injury at 28 days (FIG. 2D-F). Additionally, we quantified regional tissue kidney stiffness with atomic force microscopy, a technique we have pioneered for multiple cardiopulmonary diseases, in smooth muscle α-actin positive regions of fibrosis and the proximal tubules and found that both areas in Cdh11^{-/-} kidneys were less stiff (FIG. 2G). These data indicate that CDH11 plays an important role in the progression of AKI to CKD.

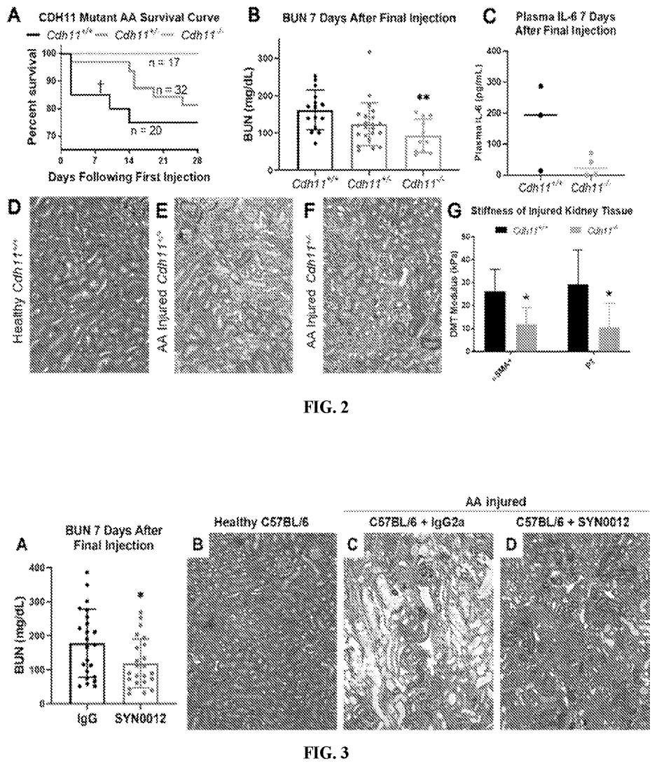

Having found that genetic deletion of Cdh11 improves survival and kidney function while also decreasing inflammatory markers, we next tested the functional blocking mAb against CDH11. SYN0012 persists in circulation for a lengthy period; early work showed that after a bolus IP injection, SYN0012 was found to have a serum half-life of ˜20 days. With the AA injury model in C57BL/6 mice, we found that SYN0012 given at 10 mg/kg on the first day of AA injection similarly reduced BUN seven days after final AA injection (FIG. 3A) and reduced fibrosis versus the IgG2a isotype control mAb at 28 days after the last AA injection (FIG. 3B-D).

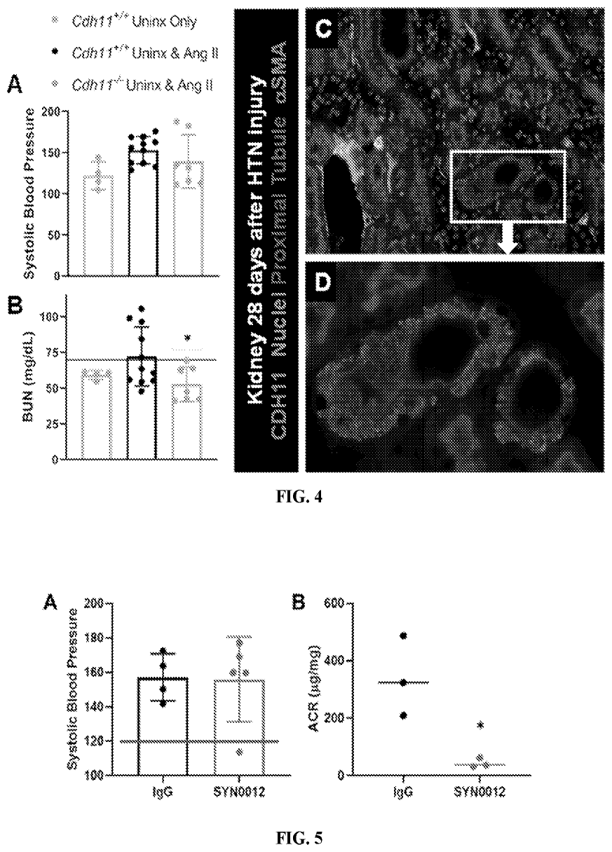

Locating CDH11 in CKD: With CDH11 identified as a biomarker of CKD and an easily accessible cell surface target for therapeutic intervention, we wanted to conclusively locate it during injury. We initially suspected that it was expressed in the interstitial space on activated myofibroblasts and was therefore driving fibrosis. However, after examining the kidneys from UUO and AA injury models, there was no evidence of CDH11 co-localizing with interstitial smooth muscle α-actin. CDH11 was also absent from the glomerulus but was very highly expressed in tubular structures of the nephron. Therefore, we utilized yet another model of kidney injury, namely a HTN model that leads to tubulointerstitial fibrosis and is achieved with a uninephrectomy and implantation of an osmotic pump with angiotensin II (AngII, 800 ng/kg/min for male mice). After four weeks, the HTN model leads to increased blood pressure and BUN in Cdh11^{+/+} animals (FIG. 4A-B) while Cdh11^{-/-} animals have increased blood pressure without increased BUN. More importantly, we were able to conclusively show overlap between CDH11 and the brush border of proximal tubules by staining with LTL (FIG. 4C-D). We also tested the CDH11 mAb preventatively in this HTN model by giving SYN0012 or IgG2a on day 0 of the HTN model. Similarly, after 28 days, blood pressure was increased in both groups (FIG. 5A), but there was a significant decrease in urine albumin-to-creatinine ratio (ACR) with the use of the CDH11 mAb (FIG. 5B).

These results have many implications. First and most importantly, prior to these data, CDH11 was recognized as a biomarker of CKD; however, our data clearly show that CDH11 is a mediator of CKD. Second, LTL expression is lost as PTECs become injured and de-differentiated, which is why we were not able to identify CDH11 co-localized with proximal tubules of the UUO and AA models—the proximal tubules were too injured and no longer expressed LTL. Third, this finding indicates that all three models of kidney injury—AA, UUO, and HTN—likely result in proximal tubule damage that leads to CDH11 expression. Fourth, we have shown in these studies that we can effectively target CDH11 to improve kidney function at the time of injury and ameliorate hallmarks of kidney fibrosis.

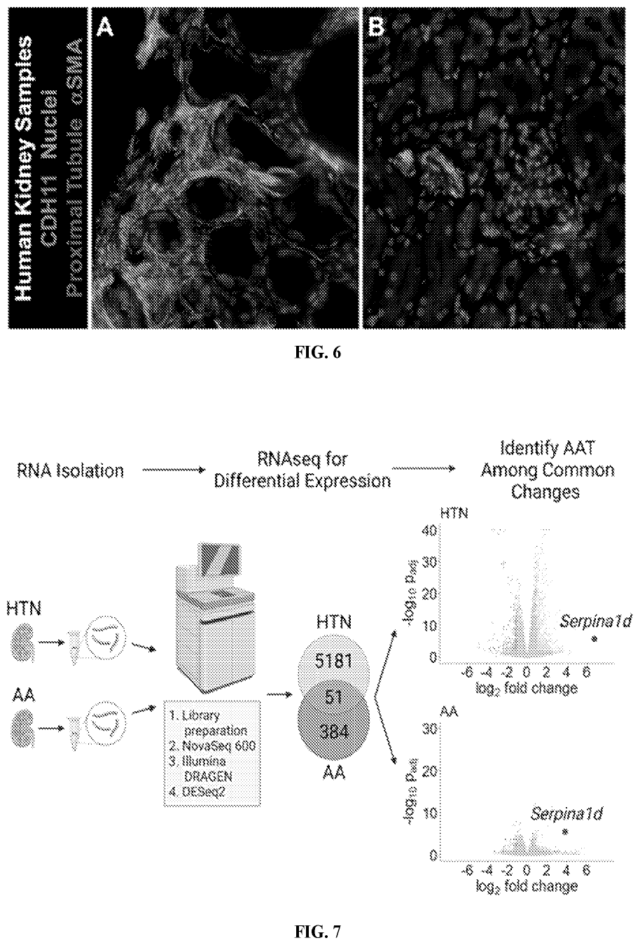

CDH11 in human kidney disease: CDH11 is a novel and unexplored potential therapeutic target for CKD. Having shown that CDH11 plays a clear role in mouse models of kidney injury, we next examined the presence of CDH11 in human kidneys with severe fibrosis. We confirmed the presence of CDH11 in the fibrotic kidney of a CKD patient (FIG. 6A) compared to negligible CDH11 expression in a healthy kidney (FIG. 6B). As in mouse models of CKD, CDH11 appears in tubular structures, presumably injured proximal tubules that no longer express the marker of healthy proximal tubules, LTL. These immunofluorescent images of human kidney disease confirm multiple findings in the mouse models we have used and propose to use in these studies. Specifically, that CDH11 is present in fibrotic kidney disease and appears to be located in tubular structures. We believe that the sum of our preliminary data in mouse kidney injury models, combined with the confirmed identification of CDH11 in human kidney disease, demonstrates that CDH11 is both a mediator and has therapeutic potential for treatment of CKD.

Determining the Mechanism by which CDH11 Engagement Between PTECs Leads to Tubulo-Interstitial Fibrosis and CKD.

Motivation: We have made significant strides in determining that CDH11 is a mediator of inflammation/fibrosis in kidney injury models; however, the mechanism of action has remained elusive. Again, though targeting CDH11 genetically or pharmacologically decreases IL-6 in the three models we have examined (UUO, AA, and HTN), we believe that this is a byproduct of there being less injury and is not a direct mechanistic outcome of targeting CDH11. As such, it is believed that engagement of CDH11 between PTECs leads to cell de-differentiation, apoptosis, and subsequent inflammation/fibrosis, which in turn leads to loss of proximal tubules and renal function. Because the mechanistic understanding of how CDH11 engagement contributes to fibrosis is unclear, we took an unbiased approach by performing RNAseq of kidneys from WT and Cdh11 null mice following AA and HTN injury models (FIG. 7). Briefly, WT and null mice were sacrificed two weeks after their last AA injection or four weeks after HTN procedure (uninephrectomy and AngII pump implantation). Analysis revealed 51 significantly changed genes common to both injury models (red=increased in Cdh11 null mice, blue=increased in WT mice), with Serpina1d being the most significant and the only gene whose protein, alpha-1-antitrypsin (AAT), appears to have any role in fibrotic disease, based on extensive literature review of all 51 genes.

Alpha-1-antitrypsin (AAT) in PTECs and CKD: AAT is a 52 kDa serine protease inhibitor that is primarily produced in the liver and is most recognized for its role in liver and lung disease. While AAT is known as a circulating hepatic stress protein, it can be produced in a variety of cell types locally, including epithelial cells, monocytes, macrophages, and neutrophils. In terms of kidney disease, there are only a handful of studies to date and these show that AAT increases rapidly in AKI, suggesting a protective effect in acute injury models to preserve PTECs, and exogenous AAT given in ischemia/reperfusion models or before tacrolimus induced injury prevents apoptosis and fibrosis.

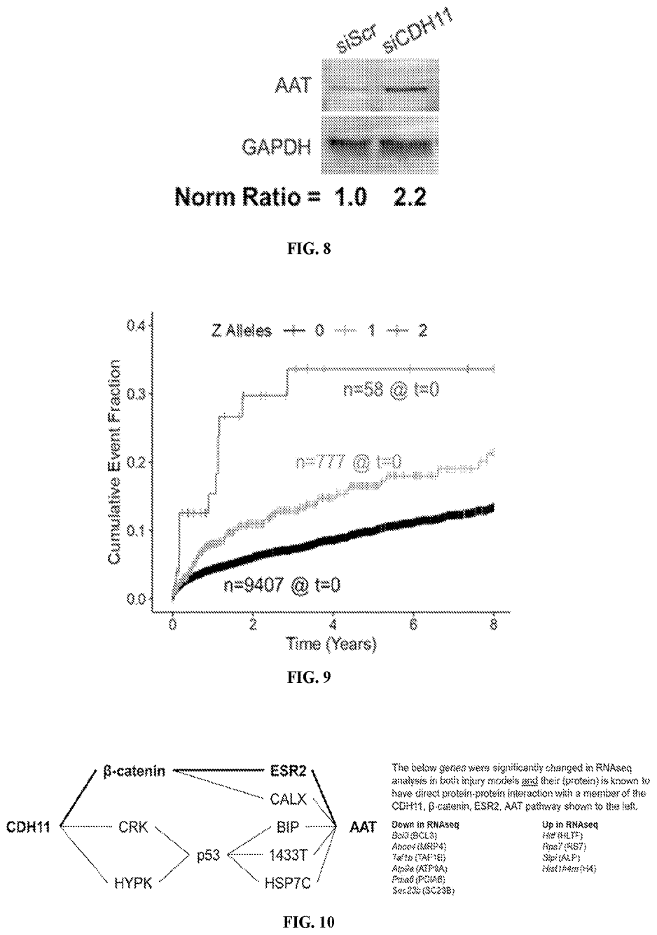

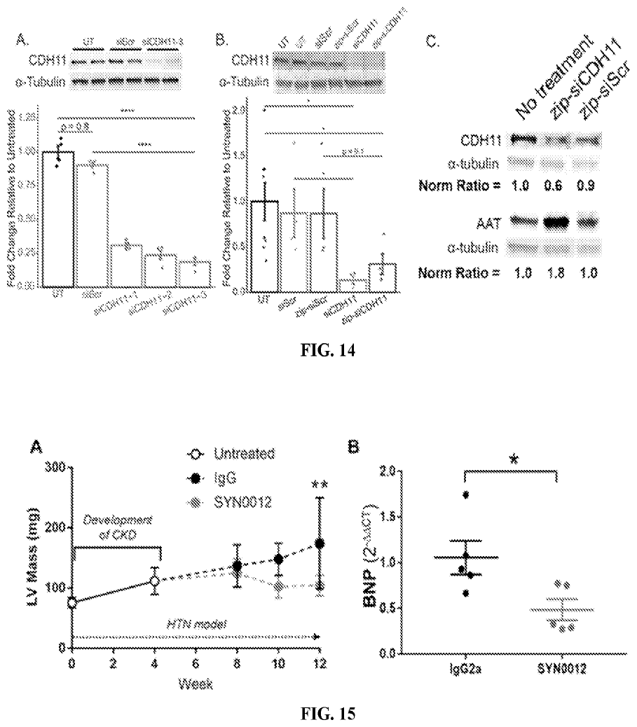

To verify that CDH11 directly alters AAT synthesis and the increase we observed in Cdh11 null mice is not due to less inflammation/fibrosis, we knocked down CDH11 in vitro with a validated siRNA (see siCDH11-3 in FIG. 14A) in immortalized PTECs. While CDH11 expression is so low in uninjured, immortalized PTECs that we could not detect it via blotting, after 72 h of CDH11 knockdown, AAT was increased 2.2-fold versus scramble control (FIG. 8). Therefore, genetic loss of CDH11 in vivo and siRNA knockdown of CDH11 in PTECs in vitro both result in increased expression of AAT.

To determine if loss of AAT function has a direct role in human cases of CKD, we used a de-identified version of the electronic medical record at Vanderbilt University Medical Center (VUMC; IRB #211049). We identified 10,242 patients who were first tested for AAT deficiency at ≥20 years of age. AAT deficiency test results were reported as the identification of two alleles. Z alleles prevent secretion and function of AAT, and patients homozygous for the Z allele typically have 10-20% the normal concentration of circulating AAT. These identities were extracted and mutant alleles quantified for regression analysis. Analysis of incident CKD was performed using a Cox proportional hazards model including age, sex, body mass index, self-reported race, ethnicity, hypertension, diabetes, and individual numbers of Z, F, and S alleles. Patients with AAT alleles other than the reference M, or mutant Z, F, or S were excluded from the study. Individual patients were censored at time of transplant of any organ, development of hepatorenal syndrome, or last recorded follow-up. CKD endpoint, censorship criteria, and disease covariates were defined by two relevant ICD codes, with the timing defined at the first occurrence. Disease covariates were included as present if they occurred at any time prior to or during the study. The incident analysis was censored at 8 years, after which less than 10% of study participants remained. Both one- and two-Z allele genotypes were associated with higher risk of CKD diagnosis in a dose-dependent fashion (FIG. 9, one Z allele: hazard ratio (HR)=1.85, 95% CI [1.44-2.38], p=1.77×10⁻⁶; two Z alleles: HR=5.35, 95% CI [3.07-9.31], p=2.97×10⁻⁹).

The data above indicates that CDH11 engagement between the PTECs leads to renal fibrosis and worsened kidney function, and it is believed that this is partially through loss of AAT expression locally. Moreover, knockdown of CDH11 leads to increased AAT in PTECs (FIG. 8) and loss of AAT is clearly associated with CKD diagnosis in patients with AAT mutations (FIG. 9).

CDH11 in PTECs: CDH11 is clearly apparent in PTECs with kidney injury, but N-cadherin is well documented to be the primary cell-cell adhesion molecule between PTECs in healthy proximal tubules. Additionally, there are multiple examples of ‘cadherin switching’ from N-cadherin to CDH11 in other cell and tissue types during fibrotic disease. When CDH11 is present, it sequesters β-catenin at the cell surface and prevents canonical Wnt/β-catenin signaling. It has been shown that β-catenin signaling is beneficial for protecting the proximal tubule from kidney fibrosis and prevents PTEC apoptosis in AKI to CKD models. It is believed that the expression of CDH11 coincides with loss of N-cadherin in PTECs and this is a key step in de-differentiation. Moreover, it is believed that persistent expression of CDH11 over time leads to PTEC apoptosis, likely through modification of canonical Wnt/β-catenin signaling.

Paracrine signaling from CDH11-engaged PTECs to immune cells: Co-culture experiments may be carried out to clarify how targeting CDH11 alters PTEC immune cell activation and activity via AAT. Briefly, pure WT, Cdh11^{-/-}, Serpina1^{-/-}, and Cdh11^{-/-}; Serpina1^{-/-} PTECs are seeded into a dish and a subset of PTECs are injured with AA or AngII 24 h later, mimicking our in vivo models. PTECs are cultured alone or with neutrophils isolated from WT bone marrow or macrophages isolated by IP injection of 1 mL of 4% thioglycollate media into WT mice, added to the PTECs 4 h after injury. It is believed that the PTECs that lack CDH11 will synthesize fewer inflammatory chemokines and co-cultures will have reduced neutrophil adhesion after 1 h due to increased AAT expression, as shown previously for lung endothelial cells treated with recombinant AAT that lacks anti-elastase activity. Moreover, PTECs lacking AAT will have increased neutrophil adhesion and, we believe, increased inflammatory cytokine synthesis signaling (e.g. IL-6 and CXCL-8).

Similarly, macrophages may be co-cultured with PTECs of different genotypes, as AAT has been shown to suppress macrophage activation into an M1-like macrophage, which is associated with inflammation and fibrosis. The macrophage-PTEC interactions may be analyzed as described above for neutrophils-PTECs. It is believed that the co-culture of macrophages with PTECs lacking CDH11 will result in less polarization of macrophages into the M1-like pro-inflammatory type, identified by markers CD80, CD86, CD64, CD16, and CD32 vs the M2-like anti-inflammatory type, identified by CD206, CD163, and CD68.

CDH11 to AAT mechanism: Because the mechanistic connection between CDH11 and AAT is unknown (PubMed returns 0 articles when any combination of CDH11 and AAT are entered), we queried HitPredict, a known protein-protein interaction database to identify the intermediate steps that may link CDH11 and AAT. Combining these known protein-protein interactions with the 51 significantly changed genes common to both AA and HTN injury models from our RNAseq datasets (FIG. 7), we were able to identify potential pathways by which CDH11 may modify AAT. Briefly, 10 of the 51 genes that were significantly changed in both models encode for proteins known to directly interact with β-catenin or estrogen receptor 2 (ESR2) proteins (FIG. 10). Thus, it is believed that the presence of CDH11 suppresses AAT synthesis via β-catenin and ESR2 linkage.

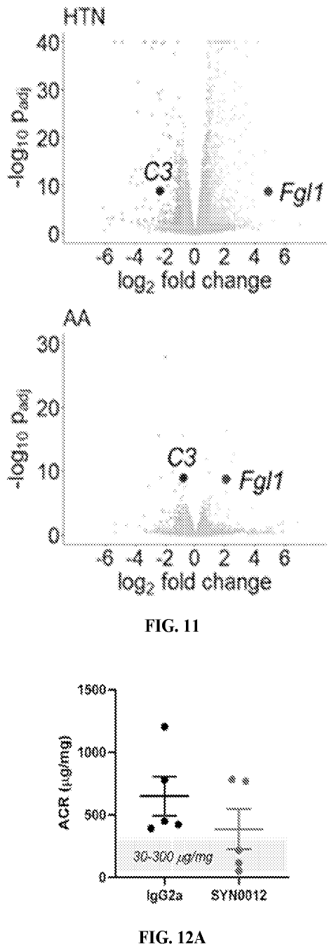

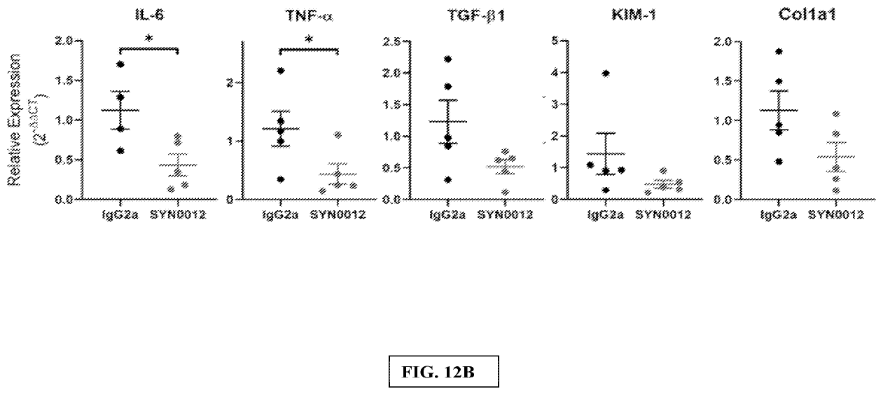

Alternative approach for Aim 1: While it is believed that CDH11 is driving PTEC de-differentiation and CKD, at least partially through AAT, there are two alternative ‘hits’ that turned up in our RNAseq data which may be assessed. Briefly, C3 was significantly decreased, while Fgl1 was significantly increased in kidneys of Cdh11 null mice (FIG. 11).

Comparing CDH11 mAb vs newly developed “zipper” modified siRNA for CDH11 (zip-siCDH11) as treatment for CKD in preclinical models of kidney injury. It is believed that CDH11 is a therapeutic target to treat (not just prevent) CKD by preventing PTEC de-differentiation and preserving AAT expression in the kidney. In the AA and HTN injury models, we will compare treatment efficacy by targeting CDH11 with a mAb that has undergone clinical trials vs. “zipper” modified siRNA for CDH11 (i.e. zip-siCDH11) that homes to the kidneys and has enhanced stabilization chemistries that provide nuclease resistance without hindering silencing activity. Thus, we additionally believe that zip-siCDH11 may be superior to mAb treatment due to the high localization of zip-siCDH11 to the kidney that we have observed in preliminary studies.

Motivation: In the above preliminary data, we tested the CDH11 mAb, SYN0012, preventatively for CKD in the UUO, AA, and HTN injury models (FIGS. 1B, 3, and 5) and had not attempted to halt/treat developed CKD. Because treatment is more challenging than prevention, we carried out a pilot study using the HTN model over 12 weeks. Briefly, male mice were given the HTN injury for 28 days, then began treatment with either IgG2a or SYN0012 (10 mg/kg) injections weekly while continuing to receive new AngII pumps monthly till they were sacrificed 8 weeks later. We observed a trend of improved renal function, as measured by ACR (FIG. 12A). Additionally, IL-6 and TNF-α were significantly decreased by CDH11 treatment, while fibrotic markers TGF-β1, KIM-1, and type I collagen (Col1a1) showed a trend to decrease (FIG. 12B). While the treatment of CKD is indeed more difficult than prevention, we believe that a more powered experiment with larger n numbers in each group and possibly with higher treatment doses of the mAb, will allow for proper assessment of treatment with a mAb for CDH11.

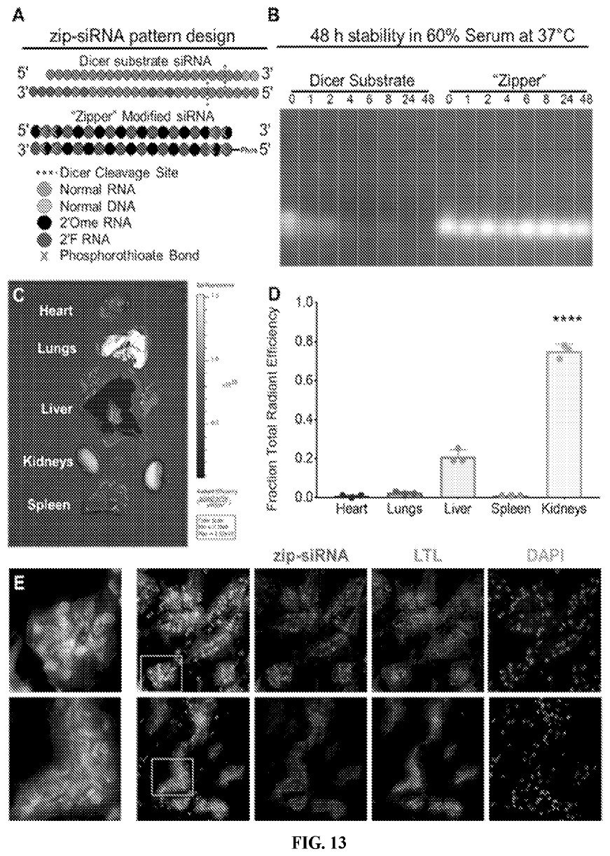

It is clear that CDH11 is a mediator of kidney fibrosis and that it can be targeted via a mAb to prevent hallmarks of CKD in multiple injury models; however, the mAb is not as efficacious as genetic deletion of Cdh11 (FIGS. 1B, 3, and 5). We speculate that this is because the mAb does not directly target the kidney, but remains in circulation for long periods of time due to the interaction of the Fc region with the recycling receptor that leads to re-adsorption in the blood stream rather than deposition in the kidney. Since zip-siRNAs do not have a re-adsorption mechanism, they accumulate heavily in the kidneys, providing for a natural drug delivery strategy to treat renal fibrosis by targeting CDH11 on PTECs. The “zipper” pattern uses alternating 2′-fluoro (2′F) and 2′-O-methyl(2′Ome) modifications on blunt-ended 19-base-pair duplex siRNA sequences (FIG. 12A), a strategy that increases serum stability while maintaining biological activity similar to the parent siRNA. Stability against exonucleases, the main culprits for endo-lysosome siRNA degradation, was achieved by adding phosphorothioate linkages at the strand ends. In preliminary studies, we tested a luciferase siRNA made with this zipper pattern and phosphorothioate modifications. In comparison to a Dicer substrate siRNA (a lightly modified design processed by Dicer for improved siRNA activity and loading that has strong precedent in the literature by the Duvall group and others), the zipper structure has dramatically superior serum stability (FIG. 13B). Moreover, this zip-siRNA homes to the kidneys of mice (FIGS. 13C-D) and is co-localized with the PTECs as indicated by LTL staining 1 h after IV injection (FIG. 13E).

We next tested (non-zip-modified) siRNAs to screen for a lead candidate siRNA sequence that most potently silences CDH11. We used two design tools (siDesign and siDIRECT) which give estimates on efficacy of siRNAs for a given open reading frame. We then looked for overlapping sequences between the two tools and chose the three sequences that were most likely to knock down CDH11 that were found with both tools. We then tested these three parent siRNA constructs (siCDH11-1, -2, or -3) in cardiac fibroblasts which express an abundance of CDH11 (FIG. 14A). Since siCHD11-3 achieved the most knockdown, we used this sequence to make the zip-siCDH11 with the stabilizing chemical modifications. The resulting siRNA, termed zip-siCDH11 has a formula:

CDH11Antisense,
5′ (PHO)(MeU)*(fA)*(MeU)(fC)(MeA)(fC)(MeA)(fA)
(MeA)(fG)(MeA)(fA)(MeU)(fU)(MeG)(fG)(MeU)*(fU)*
(MeC) 3′
CDH11Sense,
5′ (fG)*(MeA)*(fA)(MeC)(fC)(MeA)(fA)(MeU)(fU)
(MeC)(fU)(MeU)(fU)(MeG)(fU)(MeG)(fA)*(MeU)*(fA) 3′
wherein:
f = 2′F; Me = 2′-O-Me; * = PS bond; and PHO =
phosphate

We then verified that the modified zip-siCDH11 did not lose silencing ability in cardiac fibroblasts vs the parent siRNA construct (siCDH11 in FIG. 14B). To test the knockdown efficacy of the zip-siCDH11, mice were given a UUO and immediately given one of the following: no treatment, zip-siCDH11 (10 mg/kg) via retro-orbital injection, or zip-siScr (10 mg/kg) and sacrified 48 h later. We found that zip-siCDH11 decreased CDH11 expression by ˜40% while increasing AAT expression by 80% (FIG. 14C). These data demonstrate that the zip-siCDH11 construct maintains potenticy both in vitro and in vivo, and in particular knocks down CDH11 in the kidney by ˜40% in this UUO pilot study.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. United States Renal Data System. 2018.
2. Craciun F L, Bijol V, Ajay A K, Rao P, Kumar R K, Hutchinson J, Hofmann O, Joshi N, Luyendyk J P, Kusebauch U, Moss C L, Srivastava A, Himmelfarb J, Waikar S S, Moritz R L and Vaidya V S. RNA Sequencing Identifies Novel Translational Biomarkers of Kidney Fibrosis. J Am Soc Nephrol. 2016; 27:1702-13.
3. Schmidt I M, Colona M R, Kestenbaum B R, Alexopoulos L G, Palsson R, Srivastava A, Liu J, Stillman I E, Rennke H G, Vaidya V S, Wu H, Humphreys B D, Waikar S S and Kidney Precision Medicine P. Cadherin-11, Sparc-related modular calcium binding protein-2, and Pigment epithelium-derived factor are promising non-invasive biomarkers of kidney fibrosis. Kidney Int. 2021.
4. Pittet P, Lee K, Kulik A J, Meister J J and Hinz B. Fibrogenic fibroblasts increase intercellular adhesion strength by reinforcing individual OB-cadherin bonds. J Cell Sci. 2008; 121:877-86.
5 Lee D M, Kiener H P, Agarwal S K, Noss E H, Watts G F, Chisaka O, Takeichi M and Brenner M B. Cadherin-11 in synovial lining formation and pathology in arthritis. Science. 2007; 315:1006-10.
6. Valencia X, Higgins J M, Kiener H P, Lee D M, Podrebarac T A, Dascher C C, Watts G F, Mizoguchi E, Simmons B, Patel D D, Bhan A K and Brenner M B. Cadherin-11 provides specific cellular adhesion between fibroblast-like synoviocytes. The Journal of experimental medicine. 2004; 200:1673-9.
7. Clark C R, Bowler M A, Snider J C and Merryman W D. Targeting Cadherin-11 Prevents Notch1-Mediated Calcific Aortic Valve Disease. Circulation. 2017; 135:2448-2450.
8. Hutcheson J D, Chen J, Sewell-Loftin M K, Ryzhova L M, Fisher C I, Su Y R and Merryman W D. Cadherin-11 regulates cell-cell tension necessary for calcific nodule formation by valvular myofibroblasts. Arterioscler Thromb Vasc Biol. 2013; 33:114-20.
9. Bowler M A, Bersi M R, Ryzhova L M, Jerrell R J, Parekh A and Merryman W D. Cadherin-11 as a regulator of valve myofibroblast mechanobiology. Am J Physiol Heart Circ Physiol. 2018; 315: H1614-H1626.
10. Chang S K, Noss E H, Chen M, Gu Z, Townsend K, Grenha R, Leon L, Lee S Y, Lee D M and Brenner M B. Cadherin-11 regulates fibroblast inflammation. Proc Natl Acad Sci U S A. 2011; 108:8402-7.
11. Schroer A K, Bersi M R, Clark C R, Zhang Q, Sanders L H, Hatzopoulos A K, Force T L, Majka S M, Lal H and Merryman W D. Cadherin-11 blockade reduces inflammation-driven fibrotic remodeling and improves outcomes after myocardial infarction. JCI Insight. 2019; 4.
12. Daemen M A, Heemskerk V H, van't Veer C, Denecker G, Wolfs T G, Vandenabeele P and Buurman W A. Functional protection by acute phase proteins alpha (1)-acid glycoprotein and alpha (1)-antitrypsin against ischemia/reperfusion injury by preventing apoptosis and inflammation. Circulation. 2000; 102:1420-6.
13. Jeong K H, Lim J H, Lee K H, Kim M J, Jung H Y, Choi J Y, Cho J H, Park S H, Kim Y L and Kim C D. Protective Effect of Alpha 1-Antitrypsin on Renal Ischemia-Reperfusion Injury. Transplant Proc. 2019; 51:2814-2822.
14. Lim J H, Oh E J, Oh S H, Jung H Y, Choi J Y, Cho J H, Park S H, Kim Y L and Kim C D. Renoprotective Effects of Alpha-1 Antitrypsin against Tacrolimus-Induced Renal Injury. Int J Mol Sci. 2020; 21.
15. Stenvinkel P, Ketteler M, Johnson R J, Lindholm B, Pecoits-Filho R, Riella M, Heimburger O, Cederholm T and Girndt M. IL-10, IL-6, and TNF-alpha: central factors in the altered cytokine network of uremia—the good, the bad, and the ugly. Kidney Int. 2005; 67:1216-33.
16. Anavekar N S, McMurray J J, Velazquez E J, Solomon S D, Kober L, Rouleau J L, White H D, Nordlander R, Maggioni A, Dickstein K, Zelenkofske S, Leimberger J D, Califf R M and Pfeffer M A. Relation between renal dysfunction and cardiovascular outcomes after myocardial infarction. N Engl J Med. 2004; 351:1285-95.
17. Finch R, Sostelly A, Sue-Ling K, Blaeuer A, Duchateau-Nguyen G, Ukarma L, Petry C, Ravva P, Villiger P and Junker U. Results of a Phase 2 Study of Rg6125, an Anti-Cadherin-11 Monoclonal Antibody, in Rheumatoid Arthritis Patients with an Inadequate Response to Anti-Tnfalpha Therapy. Annals of the Rheumatic Diseases. 2019; 78:189-189.
18. Gewin L S. Renal fibrosis: Primacy of the proximal tubule. Matrix Biol. 2018; 68-69:248-262.
19. Takaori K, Nakamura J, Yamamoto S, Nakata H, Sato Y, Takase M, Nameta M, Yamamoto T, Economides A N, Kohno K, Haga H, Sharma K and Yanagita M. Severity and Frequency of Proximal Tubule Injury Determines Renal Prognosis. J Am Soc Nephrol. 2016; 27:2393-406.
20. Qi R and Yang C. Renal tubular epithelial cells: the neglected mediator of tubulointerstitial fibrosis after injury. Cell Death Dis. 2018; 9:1126.
21. Schneider D J, Wu M, Le T T, Cho S H, Brenner M B, Blackburn M R and Agarwal S K. Cadherin-11 contributes to pulmonary fibrosis: potential role in TGF-beta production and epithelial to mesenchymal transition. Faseb J. 2012; 26:503-12.
22. Bloodworth N C, Clark C R, West J D, Snider J C, Gaskill C, Shay S, Scott C, Bastarache J, Gladson S, Moore C, D'Amico R, Brittain E L, Tanjore H, Blackwell T S, Majka S M and Merryman W D. Bone Marrow-Derived Proangiogenic Cells Mediate Pulmonary Arteriole Stiffening via Serotonin 2B Receptor Dependent Mechanism. Circ Res. 2018; 123: e51-e64.
23. Raddatz M A, Madhur M S and Merryman W D. Adaptive immune cells in calcific aortic valve disease. Am J Physiol Heart Circ Physiol. 2019; 317: H141-H155.
24. Raddatz M A, Huffstater T, Bersi M R, Reinfeld B I, Madden M Z, Booton S E, Rathmell W K, Rathmell J C, Lindman B R, Madhur M S and Merryman W D. Macrophages Promote Aortic Valve Cell Calcification and Alter STAT3 Splicing. Arterioscler Thromb Vasc Biol. 2020; 40: e153-e165.
25. Huffstater T, Merryman W D and Gewin L S. Wnt/beta-Catenin in Acute Kidney Injury and Progression to Chronic Kidney Disease. Semin Nephrol. 2020; 40:126-137.
26. Chung S, Overstreet J M, Li Y, Wang Y, Niu A, Wang S, Fan X, Sasaki K, Jin G N, Khodo S N, Gewin L, Zhang M Z and Harris R C. TGF-beta promotes fibrosis after severe acute kidney injury by enhancing renal macrophage infiltration. JCI Insight. 2018; 3.
27. Gewin L, Bulus N, Mernaugh G, Moeckel G, Harris R C, Moses H L, Pozzi A and Zent R. TGF-beta receptor deletion in the renal collecting system exacerbates fibrosis. J Am Soc Nephrol. 2010; 21:1334-43.
28. Gewin L and Zent R. How does TGF-beta mediate tubulointerstitial fibrosis? Semin Nephrol. 2012; 32:228-35.
29. Neelisetty S, Alford C, Reynolds K, Woodbury L, Nlandu-Khodo S, Yang H, Fogo A B, Hao C M, Harris R C, Zent R and Gewin L. Renal fibrosis is not reduced by blocking transforming growth factor-beta signaling in matrix-producing interstitial cells. Kidney Int. 2015; 88:503-14.
30. Nlandu-Khodo S, Neelisetty S, Phillips M, Manolopoulou M, Bhave G, May L, Clark P E, Yang H, Fogo A B, Harris R C, Taketo M M, Lee E and Gewin L S. Blocking TGF-beta and beta-Catenin Epithelial Crosstalk Exacerbates CKD. J Am Soc Nephrol. 2017; 28:3490-3503.
31. Nlandu-Khodo S, Osaki Y, Scarfe L, Yang H, Phillips-Mignemi M, Tonello J, Saito-Diaz K, Neelisetty S, Ivanova A, Huffstater T, McMahon R, Taketo M M, deCaestecker M, Kasinath B, Harris R C, Lee E and Gewin L S. Tubular beta-catenin and Fox03 interactions protect in chronic kidney disease. JCI Insight. 2020; 5.
32. Itani H A, McMaster W G, Jr., Saleh M A, Nazarewicz R R, Mikolajczyk T P, Kaszuba A M, Konior A, Prejbisz A, Januszewicz A, Norlander A E, Chen W, Bonami R H, Marshall A F, Poffenberger G, Weyand C M, Madhur M S, Moore D J, Harrison D G and Guzik T J. Activation of Human T Cells in Hypertension: Studies of Humanized Mice and Hypertensive Humans. Hypertension. 2016; 68:123-32.
33. Kirabo A, Fontana V, de Faria A P, Loperena R, Galindo C L, Wu J, Bikineyeva A T, Dikalov S, Xiao L, Chen W, Saleh M A, Trott D W, Itani H A, Vinh A, Amarnath V, Amarnath K, Guzik T J, Bernstein K E, Shen X Z, Shyr Y, Chen S C, Mernaugh R L, Laffer C L, Elijovich F, Davies S S, Moreno H, Madhur M S, Roberts J, 2nd and Harrison D G. D C isoketal-modified proteins activate T cells and promote hypertension. J Clin Invest. 2014; 124:4642-56.
34. Madhur M S and Harrison D G. Senescent T cells and hypertension: new ideas about old cells. Hypertension. 2013; 62:13-5.
35. Trott D W, Thabet S R, Kirabo A, Saleh M A, Itani H, Norlander A E, Wu J, Goldstein A, Arendshorst W J, Madhur M S, Chen W, Li C I, Shyr Y and Harrison D G. Oligoclonal CD8+ T cells play a critical role in the development of hypertension. Hypertension. 2014; 64:1108-15.
36. Alexander M R, Norlander A E, Elijovich F, Atreya R V, Gaye A, Gnecco J S, Laffer C L, Galindo C L and Madhur M S. Human monocyte transcriptional profiling identifies IL-18 receptor accessory protein and lactoferrin as novel immune targets in hypertension. Br J Pharmacol. 2019; 176:2015-2027.
37. Dale B L, Pandey A K, Chen Y, Smart C D, Laroumanie F, Ao M, Xiao L, Dikalova A E, Dikalov S I, Elijovich F, Foss J D, Barbaro N R, Van Beusecum J P, Deger S M, Alsouqi A, Itani H A, Norlander A E, Alexander M R, Zhao S, Ikizler T A, Algood HMS and Madhur M S. Critical role of Interleukin 21 and T follicular helper cells in hypertension and vascular dysfunction. JCI Insight. 2019; 5.
38. Norlander A E, Madhur M S and Harrison D G. The immunology of hypertension. The Journal of experimental medicine. 2018; 215:21-33.
39. Norlander A E, Saleh M A, Kamat N V, Ko B, Gnecco J, Zhu L, Dale B L, Iwakura Y, Hoover R S, McDonough A A and Madhur M S. Interleukin-17A Regulates Renal Sodium Transporters and Renal Injury in Angiotensin II-Induced Hypertension. Hypertension. 2016; 68:167-74.
40. Norlander A E, Saleh M A, Pandey A K, Itani H A, Wu J, Xiao L, Kang J, Dale B L, Goleva S B, Laroumanie F, Du L, Harrison D G and Madhur M S. A salt-sensing kinase in T lymphocytes, SGK1, drives hypertension and hypertensive end-organ damage. JCI Insight. 2017; 2.
41. Saleh M A, Norlander A E and Madhur M S. Inhibition of Interleukin 17-A but not Interleukin-17F Signaling Lowers Blood Pressure and Reduces End-organ Inflammation in Angiotensin II-induced Hypertension. JACC Basic Transl Sci. 2016; 1:606-616.
42. Wu J, Montaniel K R, Saleh M A, Xiao L, Chen W, Owens G K, Humphrey J D, Majesky M W, Paik D T, Hatzopoulos A K, Madhur M S and Harrison D G. Origin of Matrix-Producing Cells That Contribute to Aortic Fibrosis in Hypertension. Hypertension. 2016; 67:461-8.
43. Wu J, Saleh M A, Kirabo A, Itani H A, Montaniel K R, Xiao L, Chen W, Mernaugh R L, Cai H, Bernstein K E, Goronzy J J, Weyand C M, Curci J A, Barbaro N R, Moreno H, Davies S S, Roberts L J, 2nd, Madhur M S and Harrison D G. Immune activation caused by vascular oxidation promotes fibrosis and hypertension. J Clin Invest. 2016; 126:50-67.
44. Evans B C, Fletcher R B, Kilchrist K V, Dailing E A, Mukalel A J, Colazo J M, Oliver M, Cheung-Flynn J, Brophy C M, Tierney J W, Isenberg J S, Hankenson K D, Ghimire K, Lander C, Gersbach C A and Duvall C L. An anionic, endosome-escaping polymer to potentiate intracellular delivery of cationic peptides, biomacromolecules, and nanoparticles. Nature communications. 2019; 10:5012.
45. Mukalel A J, Evans B C, Kilchrist K V, Dailing E A, Burdette B, Cheung-Flynn J, Brophy C M and Duvall C L. Excipients for the lyoprotection of MAPKAP kinase 2 inhibitory peptide nano-polyplexes. J Control Release. 2018; 282:110-119.
46. Hoogenboezem E N and Duvall C L. Harnessing albumin as a carrier for cancer therapies. Adv Drug Deliv Rev. 2018; 130:73-89.
47. Sarett S M, Werfel T A, Lee L, Jackson M A, Kilchrist K V, Brantley-Sieders D and Duvall C L. Lipophilic siRNA targets albumin in situ and promotes bioavailability, tumor penetration, and carrier-free gene silencing. Proc Natl Acad Sci USA. 2017; 114: E6490-E6497.
48. Bergin D A, Hurley K, McElvaney N G and Reeves E P. Alpha-1 antitrypsin: a potent anti-inflammatory and potential novel therapeutic agent. Arch Immunol Ther Exp (Warsz). 2012; 60:81-97.
49. Zager R A, Johnson A C and Frostad K B. Rapid renal alpha-1 antitrypsin gene induction in experimental and clinical acute kidney injury. PLoS One. 2014; 9: e98380.
50. Borel F, Sun H, Zieger M, Cox A, Cardozo B, Li W, Oliveira G, Davis A, Gruntman A, Flotte T R, Brodsky M H, Hoffman A M, Elmallah M K and Mueller C. Editing out five Serpina1 paralogs to create a mouse model of genetic emphysema. Proc Natl Acad Sci U S A. 2018; 115:2788-2793.
51. Prozialeck W C, Lamar P C and Appelt D M. Differential expression of E-cadherin, N-cadherin and beta-catenin in proximal and distal segments of the rat nephron. BMC Physiol. 2004; 4:10.
52. Keller C, Kroening S, Zuehlke J, Kunath F, Krueger B and Goppelt-Struebe M. Distinct mesenchymal alterations in N-cadherin and E-cadherin positive primary renal epithelial cells. PLoS One. 2012; 7: e43584.
53. Nurnberger J, Feldkamp T, Kavapurackal R, Opazo Saez A, Becker J, Horbelt M and Kribben A. N-cadherin is depleted from proximal tubules in experimental and human acute kidney injury. Histochem Cell Biol. 2010; 133:641-9.
54. Black M, Milewski D, Le T, Ren X, Xu Y, Kalinichenko V V and Kalin T V. FOXF1 Inhibits Pulmonary Fibrosis by Preventing CDH2-CDH11 Cadherin Switch in Myofibroblasts. Cell Rep. 2018; 23:442-458.
55. Koehler A, Schlupf J, Schneider M, Kraft B, Winter C and Kashef J. Loss of Xenopus cadherin-11 leads to increased Wnt/beta-catenin signaling and up-regulation of target genes c-myc and cyclin D1 in neural crest. Dev Biol. 2013; 383:132-45.
56. Gewin L, Vadivelu S, Neelisetty S, Srichai M B, Paueksakon P, Pozzi A, Harris R C and Zent R. Deleting the TGF-beta receptor attenuates acute proximal tubule injury. J Am Soc Nephrol. 2012; 23:2001-11.
57. Laroumanie F, Korneva A, Bersi M R, Alexander M R, Xiao L, Zhong X, Van Beusecum J P, Chen Y, Saleh M A, McMaster W G, Gavulic K A, Dale B L, Zhao S, Guo Y, Shyr Y, Perrien D S, Cox N J, Curci J A, Humphrey J D and Madhur M S. LNK deficiency promotes acute aortic dissection and rupture. JCI Insight. 2018; 3.
58. Jonigk D, Al-Omari M, Maegel L, Muller M, Izykowski N, Hong J, Hong K, Kim S H, Dorsch M, Mahadeva R, Laenger F, Kreipe H, Braun A, Shahaf G, Lewis E C, Welte T, Dinarello C A and Janciauskiene S. Anti-inflammatory and immunomodulatory properties of alpha1-antitrypsin without inhibition of elastase. Proc Natl Acad Sci USA. 2013; 110:15007-12.
59. Gou W, Wang J, Song L, Kim D S, Cui W, Strange C and Wang H. Alpha-1 antitrypsin suppresses macrophage activation and promotes islet graft survival after intrahepatic islet transplantation. Am J Transplant. 2021; 21:1713-1724.
1. United States Renal Data System. 2018.
2. Craciun F L, Bijol V, Ajay A K, Rao P, Kumar R K, Hutchinson J, Hofmann O, Joshi N, Luyendyk J P, Kusebauch U, Moss C L, Srivastava A, Himmelfarb J, Waikar S S, Moritz R L and Vaidya V S. RNA Sequencing Identifies Novel Translational Biomarkers of Kidney Fibrosis. J Am Soc Nephrol. 2016; 27:1702-13.
3. Schmidt I M, Colona M R, Kestenbaum B R, Alexopoulos L G, Palsson R, Srivastava A, Liu J, Stillman I E, Rennke H G, Vaidya V S, Wu H, Humphreys B D, Waikar S S and Kidney Precision Medicine P. Cadherin-11, Sparc-related modular calcium binding protein-2, and Pigment epithelium-derived factor are promising non-invasive biomarkers of kidney fibrosis. Kidney Int. 2021.
4. Pittet P, Lee K, Kulik A J, Meister J J and Hinz B. Fibrogenic fibroblasts increase intercellular adhesion strength by reinforcing individual OB-cadherin bonds. J Cell Sci. 2008; 121:877-86.
5. Lee D M, Kiener H P, Agarwal S K, Noss E H, Watts G F, Chisaka O, Takeichi M and Brenner M B. Cadherin-11 in synovial lining formation and pathology in arthritis. Science. 2007; 315:1006-10.
6. Valencia X, Higgins J M, Kiener H P, Lee D M, Podrebarac T A, Dascher C C, Watts G F, Mizoguchi E, Simmons B, Patel D D, Bhan A K and Brenner M B. Cadherin-11 provides specific cellular adhesion between fibroblast-like synoviocytes. The Journal of experimental medicine. 2004; 200:1673-9.
7. Clark C R, Bowler M A, Snider J C and Merryman W D. Targeting Cadherin-11 Prevents Notch1-Mediated Calcific Aortic Valve Disease. Circulation. 2017; 135:2448-2450.
8. Hutcheson J D, Chen J, Sewell-Loftin M K, Ryzhova L M, Fisher C I, Su Y R and Merryman W D. Cadherin-11 regulates cell-cell tension necessary for calcific nodule formation by valvular myofibroblasts. Arterioscler Thromb Vasc Biol. 2013; 33:114-20.
9. Bowler M A, Bersi M R, Ryzhova L M, Jerrell R J, Parekh A and Merryman W D. Cadherin-11 as a regulator of valve myofibroblast mechanobiology. Am J Physiol Heart Circ Physiol. 2018; 315: H1614-H1626.
10. Chang S K, Noss E H, Chen M, Gu Z, Townsend K, Grenha R, Leon L, Lee S Y, Lee D M and Brenner M B. Cadherin-11 regulates fibroblast inflammation. Proc Natl Acad Sci U S A. 2011; 108:8402-7.
11. Schroer A K, Bersi M R, Clark C R, Zhang Q, Sanders L H, Hatzopoulos A K, Force T L, Majka S M, Lal H and Merryman W D. Cadherin-11 blockade reduces inflammation-driven fibrotic remodeling and improves outcomes after myocardial infarction. JCI Insight. 2019; 4.
12. Daemen M A, Heemskerk V H, van't Veer C, Denecker G, Wolfs T G, Vandenabeele P and Buurman W A. Functional protection by acute phase proteins alpha (1)-acid glycoprotein and alpha (1)-antitrypsin against ischemia/reperfusion injury by preventing apoptosis and inflammation. Circulation. 2000; 102:1420-6.
13. Jeong K H, Lim J H, Lee K H, Kim M J, Jung H Y, Choi J Y, Cho J H, Park S H, Kim Y L and Kim C D. Protective Effect of Alpha 1-Antitrypsin on Renal Ischemia-Reperfusion Injury. Transplant Proc. 2019; 51:2814-2822.
14. Lim J H, Oh E J, Oh S H, Jung H Y, Choi J Y, Cho J H, Park S H, Kim Y L and Kim C D. Renoprotective Effects of Alpha-1 Antitrypsin against Tacrolimus-Induced Renal Injury. Int J Mol Sci. 2020; 21.
15. Stenvinkel P, Ketteler M, Johnson R J, Lindholm B, Pecoits-Filho R, Riella M, Heimburger O, Cederholm T and Girndt M. IL-10, IL-6, and TNF-alpha: central factors in the altered cytokine network of uremia—the good, the bad, and the ugly. Kidney Int. 2005; 67:1216-33.
16. Anavekar N S, McMurray J J, Velazquez E J, Solomon S D, Kober L, Rouleau J L, White H D, Nordlander R, Maggioni A, Dickstein K, Zelenkofske S, Leimberger J D, Califf R M and Pfeffer M A. Relation between renal dysfunction and cardiovascular outcomes after myocardial infarction. N Engl J Med. 2004; 351:1285-95.
17. Finch R, Sostelly A, Sue-Ling K, Blaeuer A, Duchateau-Nguyen G, Ukarma L, Petry C, Ravva P, Villiger P and Junker U. Results of a Phase 2 Study of Rg6125, an Anti-Cadherin-11 Monoclonal Antibody, in Rheumatoid Arthritis Patients with an Inadequate Response to Anti-Tnfalpha Therapy. Annals of the Rheumatic Diseases. 2019; 78:189-189
18. Gewin L S. Renal fibrosis: Primacy of the proximal tubule. Matrix Biol. 2018; 68-69:248-262.
19. Takaori K, Nakamura J, Yamamoto S, Nakata H, Sato Y, Takase M, Nameta M, Yamamoto T, Economides A N, Kohno K, Haga H, Sharma K and Yanagita M. Severity and Frequency of Proximal Tubule Injury Determines Renal Prognosis. J Am Soc Nephrol. 2016; 27:2393-406.
20. Qi R and Yang C. Renal tubular epithelial cells: the neglected mediator of tubulointerstitial fibrosis after injury. Cell Death Dis. 2018; 9:1126.
21. Schneider D J, Wu M, Le T T, Cho S H, Brenner M B, Blackburn M R and Agarwal S K. Cadherin-11 contributes to pulmonary fibrosis: potential role in TGF-beta production and epithelial to mesenchymal transition. Faseb J. 2012; 26:503-12.
22. Bloodworth N C, Clark C R, West J D, Snider J C, Gaskill C, Shay S, Scott C, Bastarache J, Gladson S, Moore C, D'Amico R, Brittain E L, Tanjore H, Blackwell T S, Majka S M and Merryman W D. Bone Marrow-Derived Proangiogenic Cells Mediate Pulmonary Arteriole Stiffening via Serotonin 2B Receptor Dependent Mechanism. Circ Res. 2018; 123: e51-e64.
23. Raddatz M A, Madhur M S and Merryman W D. Adaptive immune cells in calcific aortic valve disease. Am J Physiol Heart Circ Physiol. 2019; 317: H141-H155.
24. Raddatz M A, Huffstater T, Bersi M R, Reinfeld B I, Madden M Z, Booton S E, Rathmell W K, Rathmell J C, Lindman B R, Madhur M S and Merryman W D. Macrophages Promote Aortic Valve Cell Calcification and Alter STAT3 Splicing. Arterioscler Thromb Vasc Biol. 2020; 40: e153-e165.
25. Huffstater T, Merryman W D and Gewin L S. Wnt/beta-Catenin in Acute Kidney Injury and Progression to Chronic Kidney Disease. Semin Nephrol. 2020; 40:126-137.
26. Chung S, Overstreet J M, Li Y, Wang Y, Niu A, Wang S, Fan X, Sasaki K, Jin G N, Khodo S N, Gewin L, Zhang M Z and Harris R C. TGF-beta promotes fibrosis after severe acute kidney injury by enhancing renal macrophage infiltration. JCI Insight. 2018; 3.
27. Gewin L, Bulus N, Mernaugh G, Moeckel G, Harris R C, Moses H L, Pozzi A and Zent R. TGF-beta receptor deletion in the renal collecting system exacerbates fibrosis. J Am Soc Nephrol. 2010; 21:1334-43.
28. Gewin L and Zent R. How does TGF-beta mediate tubulointerstitial fibrosis? Semin Nephrol. 2012; 32:228-35.
29. Neelisetty S, Alford C, Reynolds K, Woodbury L, Nlandu-Khodo S, Yang H, Fogo A B, Hao C M, Harris R C, Zent R and Gewin L. Renal fibrosis is not reduced by blocking transforming growth factor-beta signaling in matrix-producing interstitial cells. Kidney Int. 2015; 88:503-14.
30. Nlandu-Khodo S, Neelisetty S, Phillips M, Manolopoulou M, Bhave G, May L, Clark P E, Yang H, Fogo A B, Harris R C, Taketo M M, Lee E and Gewin L S. Blocking TGF-beta and beta-Catenin Epithelial Crosstalk Exacerbates CKD. J Am Soc Nephrol. 2017; 28:3490-3503.
31. Nlandu-Khodo S, Osaki Y, Scarfe L, Yang H, Phillips-Mignemi M, Tonello J, Saito-Diaz K, Neelisetty S, Ivanova A, Huffstater T, McMahon R, Taketo M M, deCaestecker M, Kasinath B, Harris R C, Lee E and Gewin L S. Tubular beta-catenin and FoxO3 interactions protect in chronic kidney disease. JCI Insight. 2020; 5.
32. Itani H A, McMaster W G, Jr., Saleh M A, Nazarewicz R R, Mikolajczyk T P, Kaszuba A M, Konior A, Prejbisz A, Januszewicz A, Norlander A E, Chen W, Bonami R H, Marshall A F, Poffenberger G, Weyand C M, Madhur M S, Moore D J, Harrison D G and Guzik T J. Activation of Human T Cells in Hypertension: Studies of Humanized Mice and Hypertensive Humans. Hypertension. 2016; 68:123-32.
33. Kirabo A, Fontana V, de Faria A P, Loperena R, Galindo C L, Wu J, Bikineyeva A T, Dikalov S, Xiao L, Chen W, Saleh M A, Trott D W, Itani H A, Vinh A, Amarnath V, Amarnath K, Guzik T J, Bernstein K E, Shen X Z, Shyr Y, Chen S C, Mernaugh R L, Laffer C L, Elijovich F, Davies S S, Moreno H, Madhur M S, Roberts J, 2nd and Harrison D G. D C isoketal-modified proteins activate T cells and promote hypertension. J Clin Invest. 2014; 124:4642-56.
34. Madhur M S and Harrison D G. Senescent T cells and hypertension: new ideas about old cells. Hypertension. 2013; 62:13-5.
35. Trott D W, Thabet S R, Kirabo A, Saleh M A, Itani H, Norlander A E, Wu J, Goldstein A, Arendshorst W J, Madhur M S, Chen W, Li C I, Shyr Y and Harrison D G. Oligoclonal CD8+ T cells play a critical role in the development of hypertension. Hypertension. 2014; 64:1108-15.
36. Alexander M R, Norlander A E, Elijovich F, Atreya R V, Gaye A, Gnecco J S, Laffer C L, Galindo C L and Madhur M S. Human monocyte transcriptional profiling identifies IL-18 receptor accessory protein and lactoferrin as novel immune targets in hypertension. Br J Pharmacol. 2019; 176:2015-2027.
37. Dale B L, Pandey A K, Chen Y, Smart C D, Laroumanie F, Ao M, Xiao L, Dikalova A E, Dikalov S I, Elijovich F, Foss J D, Barbaro N R, Van Beusecum J P, Deger S M, Alsouqi A, Itani H A, Norlander A E, Alexander M R, Zhao S, Ikizler T A, Algood HMS and Madhur M S. Critical role of Interleukin 21 and T follicular helper cells in hypertension and vascular dysfunction. JCI Insight. 2019; 5.
38. Norlander A E, Madhur M S and Harrison D G. The immunology of hypertension. The Journal of experimental medicine. 2018; 215:21-33.
39. Norlander A E, Saleh M A, Kamat N V, Ko B, Gnecco J, Zhu L, Dale B L, Iwakura Y, Hoover R S, McDonough A A and Madhur M S. Interleukin-17A Regulates Renal Sodium Transporters and Renal Injury in Angiotensin II-Induced Hypertension. Hypertension. 2016; 68:167-74.
40. Norlander A E, Saleh M A, Pandey A K, Itani H A, Wu J, Xiao L, Kang J, Dale B L, Goleva S B, Laroumanie F, Du L, Harrison D G and Madhur M S. A salt-sensing kinase in T lymphocytes, SGK1, drives hypertension and hypertensive end-organ damage. JCI Insight. 2017; 2.
41. Saleh M A, Norlander A E and Madhur M S. Inhibition of Interleukin 17-A but not Interleukin-17F Signaling Lowers Blood Pressure and Reduces End-organ Inflammation in Angiotensin II-induced Hypertension. JACC Basic Transl Sci. 2016; 1:606-616.
42. Wu J, Montaniel K R, Saleh M A, Xiao L, Chen W, Owens G K, Humphrey J D, Majesky M W, Paik D T, Hatzopoulos A K, Madhur M S and Harrison D G. Origin of Matrix-Producing Cells That Contribute to Aortic Fibrosis in Hypertension. Hypertension. 2016; 67:461-8.
43. Wu J, Saleh M A, Kirabo A, Itani H A, Montaniel K R, Xiao L, Chen W, Mernaugh R L, Cai H, Bernstein K E, Goronzy J J, Weyand C M, Curci J A, Barbaro N R, Moreno H, Davies S S, Roberts L J, 2nd, Madhur M S and Harrison D G. Immune activation caused by vascular oxidation promotes fibrosis and hypertension. J Clin Invest. 2016; 126:50-67.
44. Evans B C, Fletcher R B, Kilchrist K V, Dailing E A, Mukalel A J, Colazo J M, Oliver M, Cheung-Flynn J, Brophy C M, Tierney J W, Isenberg J S, Hankenson K D, Ghimire K, Lander C, Gersbach C A and Duvall C L. An anionic, endosome-escaping polymer to potentiate intracellular delivery of cationic peptides, biomacromolecules, and nanoparticles. Nature communications. 2019; 10:5012.
45. Mukalel A J, Evans B C, Kilchrist K V, Dailing E A, Burdette B, Cheung-Flynn J, Brophy C M and Duvall C L. Excipients for the lyoprotection of MAPKAP kinase 2 inhibitory peptide nano-polyplexes. J Control Release. 2018; 282:110-119.
46. Hoogenboezem E N and Duvall C L. Harnessing albumin as a carrier for cancer therapies. Adv Drug Deliv Rev. 2018; 130:73-89.
47. Sarett S M, Werfel T A, Lee L, Jackson M A, Kilchrist K V, Brantley-Sieders D and Duvall C L. Lipophilic siRNA targets albumin in situ and promotes bioavailability, tumor penetration, and carrier-free gene silencing. Proc Natl Acad Sci USA. 2017; 114: E6490-E6497.
48. Bergin D A, Hurley K, McElvaney N G and Reeves E P. Alpha-1 antitrypsin: a potent anti-inflammatory and potential novel therapeutic agent. Arch Immunol Ther Exp (Warsz). 2012; 60:81-97.
49. Zager R A, Johnson A C and Frostad K B. Rapid renal alpha-1 antitrypsin gene induction in experimental and clinical acute kidney injury. PLoS One. 2014; 9: e98380.
50. Borel F, Sun H, Zieger M, Cox A, Cardozo B, Li W, Oliveira G, Davis A, Gruntman A, Flotte T R, Brodsky M H, Hoffman A M, Elmallah M K and Mueller C. Editing out five Serpina1 paralogs to create a mouse model of genetic emphysema. Proc Natl Acad Sci U S A. 2018; 115:2788-2793.
51. Prozialeck W C, Lamar P C and Appelt D M. Differential expression of E-cadherin, N-cadherin and beta-catenin in proximal and distal segments of the rat nephron. BMC Physiol. 2004; 4:10.
52. Keller C, Kroening S, Zuehlke J, Kunath F, Krueger B and Goppelt-Struebe M. Distinct mesenchymal alterations in N-cadherin and E-cadherin positive primary renal epithelial cells. PLoS One. 2012; 7: e43584.
53. Nurnberger J, Feldkamp T, Kavapurackal R, Opazo Saez A, Becker J, Horbelt M and Kribben A. N-cadherin is depleted from proximal tubules in experimental and human acute kidney injury. Histochem Cell Biol. 2010; 133:641-9.
54. Black M, Milewski D, Le T, Ren X, Xu Y, Kalinichenko V V and Kalin T V. FOXF1 Inhibits Pulmonary Fibrosis by Preventing CDH2-CDH11 Cadherin Switch in Myofibroblasts. Cell Rep. 2018; 23:442-458.
55. Koehler A, Schlupf J, Schneider M, Kraft B, Winter C and Kashef J. Loss of Xenopus cadherin-11 leads to increased Wnt/beta-catenin signaling and up-regulation of target genes c-myc and cyclin D1 in neural crest. Dev Biol. 2013; 383:132-45.
56. Gewin L, Vadivelu S, Neelisetty S, Srichai M B, Paueksakon P, Pozzi A, Harris R C and Zent R. Deleting the TGF-beta receptor attenuates acute proximal tubule injury. J Am Soc Nephrol. 2012; 23:2001-11.
57. Laroumanie F, Korneva A, Bersi M R, Alexander M R, Xiao L, Zhong X, Van Beusecum J P, Chen Y, Saleh M A, McMaster W G, Gavulic K A, Dale B L, Zhao S, Guo Y, Shyr Y, Perrien D S, Cox N J, Curci J A, Humphrey J D and Madhur M S. LNK deficiency promotes acute aortic dissection and rupture. JCI Insight. 2018; 3.
58. Jonigk D, Al-Omari M, Maegel L, Muller M, Izykowski N, Hong J, Hong K, Kim S H, Dorsch M, Mahadeva R, Laenger F, Kreipe H, Braun A, Shahaf G, Lewis E C, Welte T, Dinarello C A and Janciauskiene S. Anti-inflammatory and immunomodulatory properties of alpha1-antitrypsin without inhibition of elastase. Proc Natl Acad Sci USA. 2013; 110:15007-12.
59. Gou W, Wang J, Song L, Kim D S, Cui W, Strange C and Wang H. Alpha-1 antitrypsin suppresses macrophage activation and promotes islet graft survival after intrahepatic islet transplantation. Am J Transplant. 2021; 21:1713-1724.
60. Lopez Y, Nakai K and Patil A. HitPredict version 4: comprehensive reliability scoring of physical protein-protein interactions from more than 100 species. Database (Oxford). 2015; 2015.
61. Chen X H, Ruan C C, Ge Q, Ma Y, Xu J Z, Zhang Z B, Lin J R, Chen D R, Zhu D L and Gao P J. Deficiency of Complement C3a and C5a Receptors Prevents Angiotensin II-Induced Hypertension via Regulatory T Cells. Circ Res. 2018; 122:970-983.
62. Nilsson B, Hamad O A, Ahlstrom H, Kullberg J, Johansson L, Lindhagen L, Haenni A, Ekdahl K N and Lind L. C3 and C4 are strongly related to adipose tissue variables and cardiovascular risk factors. Eur J Clin Invest. 2014; 44:587-96.
63. Engstrom G, Hedblad B, Berglund G, Janzon L and Lindgarde F. Plasma levels of complement C3 is associated with development of hypertension: a longitudinal cohort study. J Hum Hypertens. 2007; 21:276-82.
64. Zhou X, Fukuda N, Matsuda H, Endo M, Wang X, Saito K, Ueno T, Matsumoto T, Matsumoto K, Soma M, Kobayashi N and Nishiyama A. Complement 3 activates the renal renin-angiotensin system by induction of epithelial-to-mesenchymal transition of the nephrotubulus in mice. Am J Physiol Renal Physiol. 2013; 305: F957-67.
65. Wang J, Sanmamed M F, Datar I, Su T T, Ji L, Sun J, Chen L, Chen Y, Zhu G, Yin W, Zheng L, Zhou T, Badri T, Yao S, Zhu S, Boto A, Sznol M, Melero I, Vignali DAA, Schalper K and Chen L. Fibrinogen-like Protein 1 Is a Major Immune Inhibitory Ligand of LAG-3. Cell. 2019; 176:334-347 e12.
66. Allerson C R, Sioufi N, Jarres R, Prakash T P, Naik N, Berdeja A, Wanders L, Griffey R H, Swayze E E and Bhat B. Fully 2′-Modified Oligonucleotide Duplexes with Improved in Vitro Potency and Stability Compared to Unmodified Small Interfering RNA. Journal of medicinal chemistry. 2005; 48:901-904.
67. Biscans A, Coles A, Echeverria D and Khvorova A. The valency of fatty acid conjugates impacts siRNA pharmacokinetics, distribution, and efficacy in vivo. Journal of Controlled Release. 2019; 302:116-125.
68. Osborn M F, Coles A H, Golebiowski D, Echeverria D, Moazami M P, Watts J K, Sena-Esteves M and Khvorova A. Efficient Gene Silencing in Brain Tumors with Hydrophobically Modified siRNAs. Molecular Cancer Therapeutics. 2018; 17:1251-1258.
69. Nair J K, Attarwala H, Sehgal A, Wang Q, Aluri K, Zhang X, Gao M, Liu J, Indrakanti R, Schofield S, Kretschmer P, Brown C R, Gupta S, Willoughby J L S, Boshar J A, Jadhav V, Charisse K, Zimmermann T, Fitzgerald K, Manoharan M, Rajeev K G, Akinc A, Hutabarat R and Maier M A. Impact of enhanced metabolic stability on pharmacokinetics and pharmacodynamics of GalNAc-siRNA conjugates. Nucleic Acids Research. 2017; 45:10969-10977.
70. Kim D-H, Behlke M A, Rose S D, Chang M-S, Choi S and Rossi J J. Synthetic dsRNA Dicer substrates enhance RNAi potency and efficacy. Nature biotechnology. 2005; 23:222-226.
71. Amarzguioui M and Rossi J J. Principles of Dicer substrate (D-siRNA) design and function. Methods Mol Biol. 2008; 442:3-10.
72. Lee Y S, Nakahara K, Pham J W, Kim K, He Z, Sontheimer E J and Carthew R W. Distinct roles for Drosophila Dicer-1 and Dicer-2 in the siRNA/miRNA silencing pathways. Cell. 2004; 117:69-81.
73. Pham J W, Pellino J L, Lee Y S, Carthew R W and Sontheimer E J. A Dicer-2-dependent 80s complex cleaves targeted mRNAs during RNAi in Drosophila. Cell. 2004; 117:83-94.
74. Orellana E A, Tenneti S, Rangasamy L, Lyle L T, Low P S and Kasinski A L. FolamiRs: Ligand-targeted, vehicle-free delivery of microRNAs for the treatment of cancer. Science translational medicine. 2017; 9.
75. Dolman M E, Harmsen S, Storm G, Hennink W E and Kok R J. Drug targeting to the kidney: Advances in the active targeting of therapeutics to proximal tubular cells. Adv Drug Deliv Rev. 2010; 62:1344-57.
76. Foster D J, Brown C R, Shaikh S, Trapp C, Schlegel M K, Qian K, Sehgal A, Rajeev K G, Jadhav V, Manoharan M, Kuchimanchi S, Maier M A and Milstein S. Advanced siRNA Designs Further Improve In Vivo Performance of GalNAc-siRNA Conjugates. Molecular Therapy. 2018; 26:708-717.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

1. A method of treating, ameliorating, or preventing a condition characterized by one or both of reduced renal function and renal fibrosis, comprising interfering with one or both of an expression and an activity of a cadherin.

2. The method of claim 1, wherein the kidney disease is chronic kidney disease (CKD).

3. The method of claim 1, wherein the interfering comprises reducing or preventing the expression of the cadherin.

4. The method of claim 1, wherein the cadherin is cadherin-11 (CDH-11).

5. The method of claim 4, wherein the reducing or preventing the expression of CDH-11 is achieved by a specific inhibition or knockdown of gene expression of CDH-11.

6. The method of claim 5 wherein the specific inhibition or knockdown of gene expression of CDH-11 is achieved by use of a silencing RNA (siRNA).

7. The method of claim 6, wherein the siRNA is modified to improve a nuclease stability.

8. The method of claim 6, wherein the modified siRNA is modified to improve one or both of an endonuclease stability and an exonuclease stability.

9. The method of claim 6, wherein in the modified siRNA phosphothorothioate bonds are substituted for phosphodiester bonds in the siRNA strand ends.

10. The method of claim 6, wherein the modified siRNA is modified at a 2′ position of a ribose ring.

11. The method of claim 10, wherein the modified siRNA is modified to include one or both of a 2′-fluoro (2′F) modification and a 2′-O-methyl(2′Ome) modification.

12. The method of claim 11, wherein the modified siRNA comprises alternating 2′-fluoro (2′F) and 2′-O-methyl(2′Ome) modifications (zip-siRNA).

13. The method of claim 12, wherein the zip-siRNA is a 19mer duplex termed zip-siCDH11 having a formula:
Antisense:
5′ (PHO)(MeU)*(fA)*(MeU)(fC)(MeA)(fC)(MeA)(fA)
(MeA)(fG)(MeA)(fA)(MeU)(fU)(MeG)(fG)(MeU)*(fU)*
(MeC) 3′
Sense:
5′ (fG)*(MeA)*(fA)(MeC)(fC)(MeA)(fA)(MeU)(fU)
(MeC)(fU)(MeU)(fU)(MeG)(fU)(MeG)(fA)*(MeU)*(fA) 3′
wherein f = 2′F; Me = 2′-O-Me; * = PS bond; and
PHO = phosphate.

14. A method of treating, ameliorating, or preventing a condition characterized by one or both of reduced renal function and renal fibrosis, comprising administering an inhibitor of cadherin expression.

15. The method of claim 14, wherein the kidney disease is chronic kidney disease (CKD).

16. The method of claim 14, wherein the cadherin is cadherin-11 (CDH-11).

17. The method of claim 14, wherein the inhibitor is a silencing RNA (siRNA).

18. The method of claim 17, wherein the siRNA is modified to improve a nuclease stability.

19. The method of claim 18, wherein the modified siRNA is modified to improve one or both of an endonuclease stability and an exonuclease stability.

20. The method of claim 18, wherein in the modified siRNA phosphothorothioate bonds are substituted for phosphodiester bonds in the siRNA strand ends.

21. The method of claim 18, wherein the modified siRNA is modified at a 2′ position of a ribose ring.

22. The method of claim 21, wherein the modified siRNA is modified to include one or both of a 2′-fluoro (2′F) modification and a 2′-O-methyl(2′Ome) modification.

23. The method of claim 22, wherein the modified siRNA comprises alternating 2′-fluoro (2′F) and 2′-O-methyl(2′Ome) modifications (zip-siRNA).

24. The method of claim 23, wherein the zip-siRNA is a 19mer duplex termed zip-siCDH11 having a formula:
Antisense:
5′ (PHO)(MeU)*(fA)*(MeU)(fC)(MeA)(fC)(MeA)(fA)
(MeA)(fG)(MeA)(fA)(MeU)(fU)(MeG)(fG)(MeU)*(fU)*
(MeC) 3′
Sense:
5′ (fG)*(MeA)*(fA)(MeC)(fC)(MeA)(fA)(MeU)(fU)
(MeC)(fU)(MeU)(fU)(MeG)(fU)(MeG)(fA)*(MeU)*(fA) 3′
wherein f = 2′F; Me = 2′-O-Me; * = PS bond; and
PHO = phosphate.

25. A method of treating, ameliorating, or preventing a condition characterized by one or both of reduced renal function and renal fibrosis, comprising administering zip-siCDH11 to a patient in need thereof in an amount sufficient to reduce or prevent cadherin-mediated suppression of alpha-1-antitrypsin (AAT) synthesis;
wherein zip-siCDH11 is a 19mer duplex termed zip-siCDH11 having a formula:
CDH11Antisense:
5′ (PHO)(MeU)*(fA)*(MeU)(fC)(MeA)(fC)(MeA)(fA)
(MeA)(fG)(MeA)(fA)(MeU)(fU)(MeG)(fG)(MeU)*(fU)*
(MeC) 3′
CDH11Sense:
5′ (fG)*(MeA)*(fA)(MeC)(fC)(MeA)(fA)(MeU)(fU)
(MeC)(fU)(MeU)(fU)(MeG)(fU)(MeG)(fA)*(MeU)*(fA) 3′
wherein f = 2′F; Me = 2′-O-Me; * = PS bond; and
PHO = phosphate.

26. The method of claim 25, wherein the condition is chronic kidney disease (CDK).
